(19) United States
(12) Patent Application Publication
(10) Pub. No.: US 2006/0223125 A1
(43) Pub. Date: Oct. 5, 2006

(54) Methods and kits for staining cell membranes

(76) Inventors: Peter I. Lelkes, Cherry Hill, NJ (US); Bahar Edrissi, Cambridge, MA (US); Elisabeth S. Papazoglou, Yardley, PA (US)

Correspondence Address: LICATA & TYRRELL P.C., 66 E. MAIN STREET, MARLTON, NJ, 08053, US

(21) Appl. No.: 11/291,568

(22) Filed: Dec. 1, 2005

Related U.S. Application Data
(60) Provisional application No. 60/632,159, filed on Dec. 1, 2004.

(51) Int. Cl. G01N 33/53

(52) U.S. Cl. 435/7.5; 977/900

(57) ABSTRACT

Methods and kits are provided for staining cell membranes using a biotinylated phospholipid in conjunction with streptavidin-coated quantum dots.

This patent application claims the benefit of priority from U.S. Provisional Application Ser. No. 60/632,159, filed Dec. 1, 2004, teachings of which are herein incorporated by reference in their entirety.

### FIELD OF THE INVENTION

The present invention relates to methods and kits for staining cell membranes with biotinylated phospholipids in conjunction with streptavidin-coated quantum dots.

### BACKGROUND OF THE INVENTION

Fluorescent dyes are used routinely in the visualization and quantitative measurement of proteins including antibodies, DNA, carbohydrates and cells. However, many of the most commonly used fluorescent dyes have characteristics that interfere with their utility. For example, many fluorescent dyes do not have a significant absorbance at the desired excitation wavelengths and are unstable in aqueous environments or changes in their environment and/or during illumination.

Fluorescent nanoparticles, referred to as quantum dots, have been used as replacements for fluorescent dyes in biological and medicinal immunoassays in biology.

Quantum dots (Qdots), also known as semiconductor nanocrystals are generally prepared with a core selected from the group consisting of Groups II-VI semiconductor materials, or Group II-V semiconductor materials. Preferred materials for the core include CdSe, CdS, or CdTe. Passivating the surface of the core quantum dot with an inorganic coating or shell such as CdS, CdSe, ZnS or ZnSe increases the quantum yield of fluorescence emission depending upon the inorganic coating used. However, these quantum dots are generally, only soluble in organic, non-polar or weakly polar solvents, thus limiting their utility in biological application involving an aqueous media.

Several attempts have been made to impart water solubility to quantum dots. For example, Chan and Nie treated water insoluble quantum dots with a large excess of mercaptocarboxylic acid in a CHCl₃ solution (Science 1998 281:2016-2018). U.S. Pat. No. 5,990,479 discloses a method for silicanizing the surface of the quantum dots to increase their water solubility.

U.S. Pat. No. 6,194,213 discloses a method for functionalizing quantum dots to be lipophillic and using these functionalized quantum dots to stain lipid membranes. The functionalized quantum dots comprise quantum dots capped with a polar capping compound and either diaminocarboxylic acid or monoaminocarboxylic acid.

Methods have also been described for use of quantum dots in binding to cell membranes via receptors. However, these methods have disadvantages in that they are dependent on receptor density and functions. Further internal labeling depends upon receptor mediated endocytosis and/or aggregation of lysosomes.

### SUMMARY OF THE INVENTION

An object of the present invention is to provide a method for staining cell membranes which comprises contacting a cell membrane with a biotinylated phospholipid in conjunction with streptavidin-coated quantum dots.

Another object of the present invention is to provide a kit for staining cell membranes which comprises biotinylated phospholipid and streptavidin-coated quantum dots.

### DETAILED DESCRIPTION OF THE INVENTION

It has now been found that bright, photostable and uniform labeling of the cell membrane is achieved using streptavidin quantum dots in conjunction with biotinylated phospholipid. This indirect technology for cell membrane staining exploits the high affinity binding of streptavidin quantum dots to a biotinylated phospholipid such as phosphoethanolamine and provides for photo-bleach resistant homogeneous staining of cells and/or cell membranes where dye internalization can occur independent of receptor mediated endocytosis.

Accordingly, one aspect of the present invention relates to a method for staining cell membranes wherein cells to be stained are contacted with a biotinylated phospholipid. Quantum dots attached to streptavidin are then added. The cell membrane outlined with the quantum dots can then be observed under a fluorescence microscope. The amount of contact time between the phospholipid/Qdot complex and the cells can be adjusted to optimize the method for different cell lines.

Using this method, quantum dots were observed to outline the cell membrane of A431 cells. Hence this method is effective in achieving cell membrane labeling. Further, the observed labeled was more uniform as compared to results using DiO or DiI where very inhomogeneous membrane cell labeling occurs. Thus, the method of the present invention overcomes disadvantages of cell membrane staining with DiO and DiI and provides for long lasting uniform fluorescence labeling.

Accordingly, the staining method of the present invention provides an improved fluorescence labeling and detection methods useful in bioassays. Further, the methods taught herein can be extended to use with other phospholipid related labeling methods outside the cell membrane. For example, methods disclosed herein could be extended to tracking movement of phospholipids attached to or related to biomolecules.

Another aspect of the present invention relates to kits for staining of the cell membranes via this method. Kits of the present invention comprise biotinylated phospholipid and streptavidin-coated quantum dots. Kits of the present invention may further comprise additional reagents, buffers and/or apparatus for use in staining of cell membranes via the method of the present invention as well as instructions for use of the kit to stain cell membranes.

The following nonlimiting examples are provided to further illustrate the present invention.

### EXAMPLES

### Example 1: Materials

1,2-Dipalmitoyl-sn-glycero-3-phosphoethanolamine-N-(biotinyl) (sodium salt; Catalog # 870285), 1,2-dioleoyl-sn-glycero-3-phosphoethanolamine-N-(biotinyl) (sodium salt; Catalog # 870282), 1,2-dipalmitoyl-sn-glycero-3-phosphoethanolamine-N-(cap biotinyl) (sodium salt; Catalog # 870277), and 1,2-dioleoyl-sn-glycero-3-phosphoethanolamine-N-(cap biotinyl) (sodium salt; Catalog # 87023) were purchased from Avanti Polar Lipids, Inc. (Alabaster, Ala.). Q-dot streptavidin conjugates (Catalog # 1014-1, 1013-1, 1011-1, 1010-1, 1012-1 and 1016-1) were purchased from Quantum Dot Corp. (Hayward, Calif.).

### Example 2: Cell Membrane Staining

A431 cells were cultured on gelatin coated 8 well Falcon culture slides. After they formed a confluent monolayer, their medium was removed and biotinylated phospholipids (Avanti Polar Lipids) of desired concentration (20 nM-2 μM) in PBS were added for 20 minutes on ice. Cells were washed twice with PBS before addition of 20 nm Streptavidin Qdot for 15-20 minutes, at temperatures varying from 23-37° C. Afterwards, cells were washed with PBS and fixed using FormaldeFresh. Cells were then observed under fluorescence microscope.

What is claimed is:

1. A method for staining a cell membrane comprising contacting a cell membrane with a biotinylated phospholipid in conjunction with streptavidin-coated quantum dots.

2. The method of claim 1 wherein the biotinylated phospholipid is a phosphoethanolamine.

3. A kit for staining cell membranes comprising biotinylated phospholipid and streptavidin-coated quantum dots.
